SEQUENCE LISTING (32 sequences)

SEQ ID NO: 1 - DNA, 20 bp, Homo sapiens
aagctgccacaagacgccaa

SEQ ID NO: 2 - DNA, 20 bp, Homo sapiens
gcctgctcatgacctcatct

SEQ ID NO: 3 - DNA, 20 bp, Homo sapiens
gcactctgctcactgtcact

SEQ ID NO: 4 - DNA, 20 bp, Homo sapiens
aagatggggctgagatgcct

SEQ ID NO: 5 - DNA, 20 bp, Homo sapiens
cacaaaaggcagctgcccaa

SEQ ID NO: 6 - DNA, 20 bp, Homo sapiens
ggtggacacacaccatggat

SEQ ID NO: 7 - DNA, 20 bp, Homo sapiens
tggcgttctactcacgtgct

SEQ ID NO: 8 - DNA, 20 bp, Homo sapiens
cagcacaggctgaggtctaa

SEQ ID NO: 9 - DNA, 20 bp, Homo sapiens
gcaacatagtgagaccccat

SEQ ID NO: 10 - DNA, 20 bp, Homo sapiens
gctatgggaatttggggagt

SEQ ID NO: 11 - DNA, 20 bp, Homo sapiens
taagagactggttcatcgaa

SEQ ID NO: 12 - DNA, 20 bp, Homo sapiens
ttaattccagcggcaggcaa

SEQ ID NO: 13 - DNA, 20 bp, Homo sapiens
gggcagcattgcaaatgcca

SEQ ID NO: 14 - DNA, 20 bp, Homo sapiens
gattgaggagcggatgcata

SEQ ID NO: 15 - DNA, 20 bp, Homo sapiens
tccgaggcgagaggctgcat

SEQ ID NO: 16 - DNA, 20 bp, Homo sapiens
cactgggaccccacttgcag

SEQ ID NO: 17 - DNA, 20 bp, Homo sapiens
gtattttagtcattttggcg

SEQ ID NO: 18 - DNA, 20 bp, Homo sapiens
agttagtgtcgggactggta

SEQ ID NO: 19 - DNA, 20 bp, Homo sapiens
gcacaacttgagctacatga

SEQ ID NO: 20 - DNA, 20 bp, Homo sapiens
ttcacagccgaagttaaagg

SEQ ID NO: 21 - DNA, 20 bp, Homo sapiens
aaaattaaaccatccttcaa

SEQ ID NO: 22 - DNA, 20 bp, Homo sapiens
ggaccatgaaacaagatgta

SEQ ID NO: 23 - DNA, 20 bp, Homo sapiens
tgagaatccagaatttgaga

SEQ ID NO: 24 - DNA, 20 bp, Homo sapiens
taatcttgagcctttttagg

SEQ ID NO: 25 - DNA, 20 bp, Homo sapiens
tcatcgtcgcaggtgcaatc

SEQ ID NO: 26 - DNA, 20 bp, Homo sapiens
aatggagaatgggaagaatc

SEQ ID NO: 27 - DNA, 20 bp, Homo sapiens
tcagtgatggagataattta

SEQ ID NO: 28 - DNA, 20 bp, Homo sapiens
ataagattaactccaccact

SEQ ID NO: 29 - DNA, 20 bp, Homo sapiens
tggcgttctactcacgtgct

SEQ ID NO: 30 - DNA, 20 bp, Homo sapiens
tttcgaagtttcagtagtac

SEQ ID NO: 31 - DNA, 20 bp, Homo sapiens
tcagtgatggagataattta

SEQ ID NO: 32 - DNA, 20 bp, Homo sapiens
tgagctgcctgtttataaat

(19) United States
(12) Patent Application Publication
(10) Pub. No.: US 2003/0165992 A1
(43) Pub. Date: Sep. 4, 2003

(54) Method for detecting a predisposition to asthma and atopy

(76) Inventors: Chaker Adra, Boston, MA (US); Taro Shirakawa, Swansea (GB); Klaus Arne Deichmann, Bollschweil (DE)

Correspondence Address: NIXON PEABODY LLP, ATTENTION: DAVID RESNICK, 101 FEDERAL STREET, BOSTON, MA, 02110, US

(21) Appl. No.: 10/127,653

(22) Filed: Apr. 22, 2002

Related U.S. Application Data
(63) Continuation of application No. 09/586,303, filed on Jun. 2, 2000, now abandoned.
(60) Provisional application No. 60/137,705, filed on Jun. 7, 1999.

(51) Int. Cl. G01N 33/53

(52) U.S. Cl. 435/7.1

(57) ABSTRACT

We have now discovered a new method for detection of a predisposition to atopy and asthma. This method involves analyzing human chromosome 5q31 for a coding variant of human IL-13, Gln110Arg.

This application claims the benefit of U.S. Provisional Application No. ______, filed ______.

### BACKGROUND OF THE INVENTION

Inflammation is a complex process in which the body's defense system combats foreign entities. While the battle against foreign entities may be necessary for the body's survival, some defense systems improperly respond to foreign entities, even innocuous ones, as dangerous and thereby damage surrounding tissue in the ensuing battle.

Atopic allergy is an ecogenetic disorder, where genetic background dictates the response to envirorunental stimuli. The disorder is generally characterized by an increased ability of lymphocytes to produce IgE antibodies in response to ubiquitous antigens. Activation of the immune system by these antigens leads to allergic inflammation and may occur after ingestion, penetration through the skin, or after inhalation. When this immune activation occurs and pulmonary inflammation ensues this disorder is broadly characterized as asthma. Certain cells are critical to this inflammatory reaction and they include T cells and antigen presenting cells. B cells that produce IgE, and mast cells/basophils and eosinophils that bind IgE. These inflammatory cells accumulate at the site of allergic inflammation and the toxic products they release contribute to the tissue destruction related to the disorder.

While asthma is generally defined as an inflammatory disorder of the airways, clinical symptoms arise from intermittent air flow obstruction. It is a chronic disabling disorder that appears to be increasing in prevalence and severity (49). It is estimated that 30-40% of the population suffers with atopic allergy, and 15% of children and 5% of adults in the population suffer from asthma (49). Thus, an enormous burden is placed on our health care resources.

The mechanism of susceptibility to atopy and asthma remains unknown. Interestingly while most individuals experience similar environmental exposures, only certain individuals develop atopic allergy and asthma. This hypersensitivity to environmental allergens, known as “atopy”, is often indicated by elevated serum IgE levels or abnormally great skin test response to allergens in atopic individuals as compared to nonatopics (50). Strong evidence for a close relationship between atopic allergy and asthma is derived from the fact that most asthmatics have clinical and serologic evidence of atopy (51). In particular, younger asthmatics have a high incidence of atopy (50). In addition, immunologic factors associated with an increase in serum total IgE levels are very closely related to impaired pulmonary function (52).

Both the diagnosis and treatment of these disorders are problematic (49). The assessment of inflamed lung tissue is often difficult, and frequently the source of the inflammation cannot be determined. Without knowledge of the source of the airwav inflammation and protection from the inciting foreign environmental agent or agents, the inflammatory process cannot be interrupted. It is now generally accepted that failure to control pulmonary inflammation leads to significant loss of lung function over time.

Current treatments suffer their own set of disadvantages. The main therapeutic agents, beta agonists, reduce the symptoms, i.e. transiently improve pulmonary functions, but do not affect the underlying inflammation so that lung tissue remains in jeopardy. In addition, constant use of beta agonists results in desensitization, which reduces their efficacy and safety (53). The agents that can diminish the underlying inflammation, the anti-inflammatory steroids, have their own known list of disadvantages that range from immunosuppression to bone loss (53).

Because of the problems associated with conventional therapies, alternative treatment strategies have been evaluated (54). Glycophorin A (55), cyclosporin (54), and a non-peptide fragment of IL-2 (56), all inhibit interleukin-2 dependent T lymphocyte proliferation and, therefore, IL-9 production (57); however, they are known to have many other effects (53). For example, cyclosporin is used as a immunosuppressant after organ transplantation. While these agents may represent alternatives to steroids in the treatment of asthmatics (56), they inhibit interleukin-2 dependent T lymphocyte proliferation and potentially critical immune functions associated with homeostasis. What is needed in the art is the identification of a pathway critical to the development of asthma that explains the episodic nature of the disorder and the close association with allergy that is downstream of these critical immune functions. Nature demonstrated that this pathway is the appropriate target for therapy since biologic variability normally exists at this pathway and these individuals are otherwise generally not immunocompromised or ill, except for their symptoms of atopy.

Because of the difficulties related to the diagnosis and treatment of asthma, the complex pathophysiology of this disorder is under intensive study. Although this disorder is heterogeneous and may be difficult to define because it can take many forms, certain features are found in common among asthmatics. Examples of such traits include elevated serum IgE levels, abnormal skin test response to allergen challenge, bronchial hyperresponsiveness [BHR], bronchodilator reversibility, and airflow obstruction (52). These expressions of these asthma related phenotypes may be studied as quantitative or qualitative measures.

Elevated IgE levels are also closely correlated with BHR, a heightened bronchoconstrictor response to a variety of stimuli (51). BHR is believed to reflect the presence of airway inflammation (58), and is considered a risk factor for asthma (59). BHR is accompanied by bronchial inflammation and an allergic diathesis in asthmatic individuals (60). Even in children with no symptoms of atopy and asthma, BHR is strongly associated with elevated IgE levels (61).

A number of studies document a heritable component to atopy and asthma (51). However, family studies have been difficult to interpret since these disorders are significantly influenced by age and gender, as well as many environmental factors such as allergens, viral infections, and pollutants (62). Moreover, because there is no known biochemical defect associated with susceptibility to these disorders, the mutant genes and their abnormal gene products can only be recognized by the anomalous phenotypes they produce. Thus, an important first step in isolating and characterizing a heritable component is identifying the chromosomal locations of the genes.

Cookson et al. provided the first description of a genetic localization for inherited atopy (63). These investigators described evidence for genetic linkage between atopy and a single marker on a specific chromosomal region designated 11q13.1. Later, they suggested evidence of maternal inheritance for atopy at this locus (64). Although maternal inheritance had been observed for atopy, it had never been explained previously. However, efforts to confirm this linkage have not been generally successful (65).

Recently, the P subunit of the high-affinity IgE receptor was mapped to chromosome 11q, and a putative mutation associated with atopy has been described in this gene (38). However, because of the difficulties by others of replicating this linkage, the significance of this gene and polymorphism remains unclear. While additional studies will be required to con firm whether this putative mutation causes atopy in the general population, data collected so far suggests this polymorphism is unlikely to represent a frequent cause of atopy.

Because serum IgE levels are so closely associated with the onset and severity of allergy and asthma as clinical disorders, attention has focused on studies of the genetic regulation of serum total IgE levels. While past studies have provided evidence for Mendelian inheritance for serum total IgE levels (66), an indication of the existence of one regulatory gene, others have found evidence for polygenic inheritance of IgE, i.e. existence of several responsible genes (67).

Artisans have found several genes that may be important in the regulation of IgE and the development or progression of bronchial inflammation associated with asthma on chromosome 5q. They include genes encoding several interleukins, such as IL-3, IL-4, IL-5, IL-9, IL-13, granulocyte macrophage colony stimulating factor [GM CSF], a receptor for macrophage colony stimulating factor [CSF-IR], fibroblast growth factor acidic [FGFA], as well as others (68). Recent evidence from family studies suggests genetic linkage between serum IgE levels and DNA markers in the region of these candidate genes on chromosome Sq (69). Together, these investigations suggest that one or more major genes in the vicinity of the interleukin complex on chromosome 5q regulate a significant amount of the observed biologic variability in serum IgE that is likely to be important in the development of atopy and asthma.

Linkage [sib-pair analyses] was also used previously to identify a genetic localization for BHR (70). Because BHR was known to be associated with a major gene for atopy, chromosomal regions reported to be important in the regulation of serum IgE levels were examined (71).

Candidate regions for atopy have been identified by linkage analyses. These studies identified the existence of a major gene for atopy on human chromosome 5q31-q33 (71).

IL-13 is a 12 kDa protein product and shares several biological profiles with IL-4 (1.2), including IgE production, CD23 and MHC class II expression, inhibition of antibody-dependent cell-mediated cytotoxicity with downregulation of IgG type I receptor (Fc[gamma]R1), and suppression of type I interferon. Although IL-4 and IL-13 possess many similar biological activities (1, 2), IL-13 shows some unique activities. Unlike IL-4-deficient mice, IL-13-null mice fail to generate goblet cells, responsible for mucus overproduction in asthma, fail to recover basic IgE levels after stimulation with IL-4 and fail to expel helminths (7). IL-13 operates through IL-13R, a heterodimer of IL-4R[alpha] and IL-13R[alpha] chains (1-3). Transgenic mice, with the promoter of the Clara cell 10 kDa protein (CC10) driving IL-13 expression selectively in the lungs, exhibit BHR to methacholine in addition to bronchial eosinophil prominence, epithelial cell hyperplasia, mucus cell metaplasia hyperproduction of mucus, deposition of Charcot-Leyden-like crystals and subepithelial airway fibrosis (8); these features are typical of Tₕ2 inflammation-induced asthmatic airways. These findings suggest that IL-13 is crucial for allergen-induced BHR in animal models, and may be relevant to human asthma (9, 10). Significantly higher IL-13 levels have been found in asthmatic patients with and without atopy (11, 12). One report has related a polymorphism within the promoter region of IL-13 with allergic asthma in a Dutch population (13).

There is a specific need in the art for genetic information on atopic allergy, asthma, and bronchial hyperresponsiveness, and for elucidation of the role of IL-13 in the etiology of these disorders, as well as a need to identify individuals who could be sufficiently treated by certain drugs. There is also a need for locating additional and more accurate markers that are predictive of susceptibility to atopy and asthma.

### SUMMARY OF THE INVENTION

We have now discovered a new method for detection of a predisposition to atopy and asthma. This method involves analyzing human chromosome 5q31 for a coding variant of human IL-13, Gln110Arg.

Early determination of a predisposition is extremely important because of the environmental effects on atopy and asthma. By knowing of such susceptibility, the individual can avoid environmental triggers. Additionally, knowledge of such risk of susceptibility permits earlier medical intervention.

Furthermore, the method of the present invention allows the patients physician to more precisely prescribe drugs in treating the disease, e.g. IL-13 inhibitors. Such drugs may be obtained using the coding variant of IL-13 in a drug screening assay.

The present invention further provides a nucleic acid containing a coding variant of human IL-13, Gln110Arg. The nucleic acid is used to produce the encoded protein, which may be employed for functional studies, in studying associated physiological pathways and for the production of antibodies. The nucleic acid compositions and antibodies specific for the protein are useful as diagnostics to identify a hereditary predisposition to atopy and asthma in a patient.

### DETAILED DESCRIPTION OF THE INVENTION

We have now discovered a novel coding variant of the human IL-13, Gln110Arg on chromosome 5q31. Gln110Arg shows association with clinical asthma across different ethnic populations, including atopic (high IgE levels) and non-atopic asthma (Table 1): it associates with higher serum IL-13 levels.

The provided IL-13 gene variant and fragments thereof, encoded protein, and antibodies thereto are useful in the identification of individuals predisposed to development of atopy and asthma. The encoded IL-13 variant is useful as an immunogen to raise specific antibodies, in drug screening for compositions that modulate IL-13 activity or expression of the protein.

Asthma, as defined herein, is reversible airflow limitation in a patient over a period of time. The disease is characterized by increased airway responsiveness to a variety of stimuli, and airway inflammation. A patient diagnosed as asthmatic will generally have multiple indications overtime, including wheezing, asthmatic attacks, and a positive response to methacholine challenge, i.e. a PC₂₀, on methacholine challenge of less than about 4 mg/ml. Guidelines for diagnosis may be found in the National Asthma Education Program Expert Panel—guidelines for diagnosis and management of asthma, National Institutes of Health, Pub. #91-3042 (1991). Atopy, respiratory infection and environmental predisposing factors may also be present, but are not necessary elements of an asthma diagnosis. Asthma conditions strictly related to atopy are referred to as atopic asthma.

The IL-13 variant locus has been mapped to human chromosome 5q31. An SSCP analysis among more than 200 atopic subjects identified different electrophoretic patterns in exon 4 of IL-13; subsequent direct sequencing identified an A4464 variant (18), indicating replacement of arginine (Arg) by glutamine (Gln) at position 110 of the mature protein. Genetic association was found between the variant and clinical asthma and increased IgE levels in two populations (Table 1).

The DNA sequence containing the coding of the human IL-13 variant may be cDNA or genomic DNA, or a fragment thereof. The term “IL-13 variant gene” shall be intended to mean a gene encoding an IL-13 Gln110Arg coding variant. The gene may be introduced into an appropriate vector for extrachromosomal maintenance or for integration into the host.

The terms “variant”, “IL-13 variant” or “Gln110Arg IL-13” as used herein are intended to mean the protein encoded by human IL-13 Gln110Arg.

The term “cDNA” as used herein is intended to include all nucleic acids that share the arrangement of sequence elements found in native mature mRNA species, where sequence elements are exons and 3′ and 5′ non-coding regions. Normally, mRNA species have contiguous exons, with the intervening introns removed by nuclear RNA splicing, to create a continuous open reading frame encoding the IL-13 variant protein.

The nucleic acid compositions of the subject invention may encode all or a part of the subject polypeptides. Fragments may be obtained of the DNA sequence by chemically synthesizing oligonucleotides in accordance with conventional methods, by restriction enzyme digestion, by PCR amplification, etc. For the most part, DNA fragments will be of at least 15 nt, usually at least 18 nt, more usually at least about 50 nt. Such small DNA fragments are useful as primers for PCR, hybridization screening, etc. Larger DNA fragments, i.e. greater than 100 nt, are useful for production of the encoded polypeptide. For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other.

The IL-13 variant gene is isolated and obtained in substantial purity, generally as other than an intact mammalian chromosome. Usually, the DNA will be obtained substantially free of other nucleic acid sequences that do not include an IL-13 variant sequence or fragment thereof, generally being at least about 50%, usually at least about 90% pure, and are typically “recombinant”, i.e. flanked by one or more nucleotides with which it is not normally associated on a naturally occurring chromosome.

The DNA sequences are used in a variety of ways in the methods of the present invention. For example, the DNA may be used to identify expression of the gene in a biological specimen. The manner in which one probes cells for the presence of particular nucleotide sequences, as genomic DNA or RNA, is well established in the literature and does not require elaboration here, mRNA is isolated from a cell sample. mRNA may be amplified by RT-PCR, using reverse transcriptase to form a complementary DNA strand, followed by polymerase chain reaction amplification, using primers specific for the subject DNA sequences. Alternatively, mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridizations, and hybridization to DNA probes arrayed on a solid chip may also find use. Detection of mRNA hybridizing to the subject sequence is indicative of IL-13 variant gene expression in the sample.

The DNA may also be employed for synthesis of a complete Gln110Arg IL-13 or polypeptide fragments thereof, particularly fragments corresponding to functional domains, binding sites, etc., and including fusions of the subject polypeptides to other proteins or parts thereof. For expression, an expression cassette may be employed, providing for a transcriptional and translational initiation region, which may be inducible or constitutive, where the coding region is operably linked under the transcriptional control of the transcriptional initiation region, and a transcriptional and translational termination region. Various transcriptional initiation regions may be employed that are functional in the expression host.

The polypeptides may be expressed in prokaryotes or eukaryotes in accordance with conventional ways, depending upon the purpose for expression. For large scale production of the protein, a unicellular organism, such as E. coli, B. subtilis, S. cerevisiae, or cells of a higher organism such as vertebrates, particularly mammals, e.g. COS 7 cells, may be used as the expression host cells. In many situations, it may be desirable to express the Gln110Arg IL-13 in mammalian cells, where the protein variant gene will benefit from native folding and post-translational modifications. Small peptides can also be synthesized in the laboratory.

With the availability of the polypeptides in large amounts, by employing an expression host, the polypeptides may be isolated and purified in accordance with conventional ways. A lysate may be prepared of the expression host and the lysate purified using HPLC, exclusion chromatography, gel electrophoresis, affinity chromatography, or another purification technique. The purified polypeptide will generally be at least about 80% pure, preferably at least about 90% pure, and may be up to and including 100% pure. Pure is intended to mean free of other proteins, as well as cellular debris.

The polypeptide is used for the production of antibodies, where short fragments provide for antibodies specific for the particular polypeptide, and larger fragments, or the entire protein, allow for the production of antibodies over the surface of the polyeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes are immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds. Cold Spring Harbor Laboratories, Cold Spring Harbor, N.Y. (1988). If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage “display” libraries, usually in conjunction with in vitro affinity maturation.

Detection of susceptibility of an individual for asthma is performed by protein, DNA or RNA sequence and/or hybridization analysis of any convenient sample from a patient, e.g. biopsy material, blood sample, scrapings from cheek, etc. A nucleic acid sample from a patient having asthma that may be associated with the coding variant Gln110Arg IL-13, is analyzed for the presence of the Gln110Arg polymorphism in the IL-13 gene The presence of Gln110Arg polymorphic IL-13 sequence is considered a predisposing polymorphism. Individuals are screened by analyzing their DNA or mRNA for the presence of a predisposing polymorphism, as compared to an asthma neutral sequence.

Screening may also be based on the antigenic characteristics of the protein. Immunoassays designed to detect the Gln110Arg variation in IL-13 proteins may be used in screening.

A number of methods are available for analyzing nucleic acids for the presence of a specific sequence. Where large amounts of DNA are available, genomic DNA is used directly. Alternatively, the region of interest is cloned into a suitable vector and grown in sufficient quantity for analysis. Cells that express IL-13 genes may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction amplification may also be used to determine whether the coding variant is present, by using a primer that is specific for the coding variant. Alternatively, various methods are known in the art that utilize oligonucleotide ligation as a means of detecting polymorphisms; for examples, see Riley et al (1990) N.A.R. 18:2887-2890 and Delahunty et al (1996) Am. J. Hum. Genet. 68:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H, etc. The label may be a two stage system, where the amplified DNA is conjugated to biotin, haptens, etc., having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods, and the sequence of bases compared to a neutral IL-13 sequence. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility.

The hybridization pattern of a control and variant sequence to an array of oligonucleotide probes immobilized on a solid support, as described in U.S. Pat. No. 5,445,934, may be used as a means of detecting the presence of the variant sequence.

Antibodies specific for Gln110Arg IL-13 of the IL-13 variants may be used in screening immunoassays. A sample is taken from a patient suspected of having IL-13 variant-associated asthma. Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. Biopsy samples are of particular interest, e.g. trachea scrapings, etc. The number of cells in a sample will generally be at least about 10³, usually at least 10⁴, more usually at least about 10⁵. The cells may be dissociated, in the case of solid tissues, or tissue sections may be analyzed. Alternatively, a lysate of the cells may be prepared.

Diagnosis may be performed by a number of methods. The different methods all determine the absence or presence or altered amounts of normal or IL-13 variant in patient cells. For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluoresces, chemiluminescers, or other labels for direct detection. Alternatively, a second stage antibody or reagent is used to amplify the signal. Such reagents are well known in the art. For example, the primary antibody may be conjugated to biotin, with horseradish peroxidase conjugated avidin added as a second stage reagent. Final detection uses a substrate that undergoes a color change in the presence of the peroxidase. The absence or presence of antibody binding may be determined by various methods including flow cytometry of dissociated cells, microscopy, radiography, scintillation counting, etc.

After the second binding step, the insoluble support is again washed free of non-specifically bound material. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for IL-13 as desired, conveniently using a labeling method as described for the sandwich assay.

The subject nucleic acids can be used to generate genetically modified non-human animals or site specific gene modifications in cell lines. The term “transgenic” is intended to encompass genetically modified animals having an exogenous IL-13 variant gene that is stably transmitted in the host cells. Transgenic animals may be made through homologous recombination, where the IL-13 locus is altered. Alternatively, a nucleic acid construct is randomly integrated into the genome. Vectors for stable integration include plasmids, retroviruses and other animal viruses, YACS, and the like. Of interest are transgenic mammals, e.g. cows, pigs, goats, horses, etc., and particularly rodents, e.g. rats, mice, etc.

The exogenous gene is usually either from a different species than the animal host, or is otherwise altered in its coding sequence to encode the Gln110Arg variant. The introduced gene is usually operably linked to a promoter, which may be constitutive or inducible, and other regulatory sequences required for expression in the host animal.

The modified cells or animals are useful in the study of IL-13 variant function and regulation. Animals may be used in functional studies, drug screening, etc., e.g. to determine the effect of a candidate drug on asthma.

By providing for the production of large amounts of the IL-13 variant, one can identify ligands or substrates that bind to, modulate or mimic the action of the IL-13 variant. Areas of investigation are the development of asthma treatments. Drug screening identifies agents that block or inhibit variant function in affected cells. Conversely, agents that reverse or inhibit variant function may stimulate bronchial reactivity. Of particular interest are screening assays for agents that have a low toxicity for human cells. A wide variety of assays may be used for this purpose, including labeled in vitro protein-protein binding assays, protein-DNA binding assays, electrophoretic mobility shift assays, immunoassays for protein binding, and the like. The purified protein may also be used for determination of three-dimensional crystal structure, which can be used for modeling intermolecular interactions, transcriptional regulation, etc.

The term “agent” as used herein describes any molecule, e.g. protein or pharmaceutical, with the capability of altering the physiological function of the variant. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of S synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc., may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The compounds having the desired pharmacological activity may be administered in a physiologically acceptable carTier to a host for treatment of asthma attributable to the IL-13 variant. The therapeutic agents may be administered in a variety of ways, orally, topically, parenterally, e.g. subcutaneously, intraperitoneally, by viral infection, intravascularly, etc. Inhaled treatments are of particular interest. Depending upon the mainer of introduction, the compounds may be formulated in a variety of ways. The concentration of therapeutically active compound in the formulation may vary from about 0.1-100wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils, and fats. Stabilizing agents, wetting and emulsifying agents, salts for varying the osmotic pressure or buffers for securing an adequate pH value, and skin penetration enhancers can be used as auxiliary agents.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for.

### EXAMPLE 1

Materials and Methods

Populations Studied and Phenotypes

Three distinct populations were studied, comprising a total of 890 subjects to whom uniform criteria were applied for assignment as clinical asthma or atopy: (i) the British case-control group included 150 young adult subjects with clinical asthma and atopy, and 150 healthy controls, all from the Oxford region (39); (ii) the Japanese case-control group included 100 young adults with clinical asthma and atopy, 100 adults with clinical asthma but no atopy (non-atopic or intrinsic asthma) and 100 young adults attending a ‘well man’ clinic as controls, all drawn from the Osaka region (40); (iii) a general childhood Japanese population of 290 schoolchildren from Wakayama Prefecture who showed negative tuberculin responses at 6 and 12 years old, of whom 46.8% showed atopy and 13.4% clinical asthma (19). All the asthmatic subjects had specialist physician-diagnosed asthma with: (i) recurrent breathlessness and chest tightness requiring on-going treatment; (ii) physician-documented wheeze; and (iii) documented labile airflow obstruction with variability in serial peak expiratory flow rates >30%. There were no heavy smokers (>20 cigarettes/day) among the subjects. Allergen-specific IgE (ASE) was detected by the CAP ELISA system (Pharmacia, Uppsala, Sweden) and the criteria for a positive titre were as used previously (39, 40). A high IgE titre (CAP system) was taken as>2 SD above published normal values (39, 40). Atopy was defined as high IgE levels, by the presence of a high concentration of total serum IgE, or a positive ASE against one or more highly purified aero-allergens.

SSCP analysis and direct sequencing

SSCP analysis for human IL-13 and IL-13R[alpha]1 was done as follows: searching for polymorphisms in the four exons of IL-13, we used the following primers: 5′-AAG CTG CCA CAA GAC GCC AA-3′(SEQ ID NO:1) and 5′-GCC TGC TCA TGA CCT CAT CT-3′(SEQ ID NO:2) for exon 1; 5′-GCA CTC TGC TCA CTG TCA CT-3′(SEQ ID NO:3) and 5′-AAG ATG GGG CTG AGA TGC CT-3′(SEQ ID NO:4) for exon 2; 5′-CAC AAA AGG CAG CTG CCC AA-3′(SEQ ID NO:5) and 5′-GGT GGA CAC ACA CCA TGG AT-3′(SEQ ID NO:6) for exon 3; and 5′-TGG CGT TCT ACT CAC GTG CT-3′(SEQ ID NO:7) and 5′-CAG CAC AGG CTG AGG TCT AA-3′ (SEQ ID NO:8) for exon 4. The annealing temperature was 60° C. in all cases.

Primers for the IL-13 promoter region were: 5′-GCA ACA TAG TGA GAC CCC AT-3′ (SEQ ID NO:9) and 5′-GCT ATG GGA ATT TGG GGA GT-3′(SEQ ID NO:10); 5′-TAA GAG ACT GOT TCA TCG AA-3′(SEQ ID NO:11) and 5′-TTA AT T CCA GCG GCA GGC AA-3′(SEQ 25 ID NO:12); and 5′-GGG CAG CAT TGC AAA TGC CA-3′(SEQ ID NO: 13) and 5′-GAT TGA GGA GCG GAT GCA TA-3′(SEQ ID NO:14). These combinations of primers covered 739 bp of sequence upstream from the ATG codon. The annealing temperature for the first set was 63° C., whereas that for the latter two was 55° C. When searching for polymorphisms in the IL-13R[alpha] 1 seven sets of primers were used for SSCP: 5′-TCC GAG GCG AGA GGC TGC AT-3′ (SEQ ID NO:15) and 5′-CAC TGG GAC CCC ACT TGC AG-3′ (SEQ ID NO:16) (60° C.); 5′-GTA TTT TAG TCA TTT TGG CG-3′ (SEQ ID NO: 17) and 5′-AGT TAG TGT CGG GAC TGG TA-3′ (SEQ ID NO: 18) (60° C.); 5′-GCA CAA CTT GAG CTA CAT GA-3′ (SEQ ID NO:19) and 5′-TT CAC AGC CGA AGT TAA AGG-3′ (SEQ ID NO:20) (60° C.); 5′-AAA ATT AAA CCA TCC TTC AA-3′(SEQ ID NO:21) and 5′-GGA CCA TGA AAC AAG ATG TA-3′ (SEQ ID NO:22) (50° C.); 5′-TGA GAA TCC AGA ATT TGA GA-3′ (SEQ ID NO:23) and 5′-TAA TCT TGA GCC TTT TTA GG-3′ (SEQ ID NO:24) (45° C.); 5′-TCA TCG TCG CAG GTG CAA TC-3′ (SEQ ID NO:25) and 5′-AAT GGA GAA TGG GAA GAA TC-3′ (SEQ ID NO:26) (50° C.); and 5′-TCA GTG ATG GAG ATA ATT TA-3′ (SEQ ID NO:27) and 5′-ATA AGA TTA ACT CCA CCA CT-3′(SEQ ID NO:28) (55° C.). The annealing temperature is shown in parentheses. The amplified products were resolved on non-denaturing polyacrylamide gels under four different conditions: 10% polyacrylamide gel containing 0 or 10% glycerol at 10 or 20° C. for 2 h. The gels were visualized by silver staining. Sequencing was conducted with the ‘big-dye system’ (Applied Biosystems, Warrington, UK) using downstream primers for IL-13 and IL-13R[alpha]1, and the image was visualized in the commercial POP-6 gel using an automated sequencer (ABI Prism 310 Genetic Analyser).

Serum IL-13 Assay

Serum IL-13 was immunoassayed in the Mitsubishi Kagaku BCL laboratories by means of a commercial kit (19). To limit circadian variation in cytokine production, blood samples were obtained between 09:00 and 10:00 h. The minimal detectable level was 3.1 pg/ml.

Genotypinf

DNA samples were extracted using the IsoQuick kit (Microprobe, Garden Grove, Ill.). For genotyping Gln110Arg of IL-13, PCR primers were: 5′-TGG CGT TCT ACT CAC GTG CT-3′ (SEQ ID NO:29) and 5′-TTT CGA AGT TTC AGT AGI AC-3′ (SEQ ID NO:30) (underlined bases were mutated to incorporate a restriction site). Amplified products were digested with Scal at 25° C. For genotyping IL-13R[alpha] 1, primers were: 5′-TCA GTG ATG GAG ATA ATT TA-3′ (SEQ ID NO:31) and 5′-TGA GCT GCC TGT TTA TAA AT-3′ (SEQ ID NO:32). Amplification products were digested with MseI. Genotyping IleSOVal and Arg551Gln of the IL-4R[alpha] (16), −590C/T promoter of the IL-4 (41) was as described elsewhere.

Computer Modeling

To assess the effect of replacement at position 110 on the internal constitution of IL-3 ligand, the modeling was conducted using the Homology module of the graphic program Insight 98 (Biosym, 1998) on a Silicon Graphic OCTANE workstation. The co-ordinate of 2.25 A crystal structure of IL-4 (Protein Data Bank accession no. 1rcb) was used as a template for homology modeling of IL-13. The Gln110Arg variant of the IL-13 was built up using the Biopolymer module on the basis of IL-13 model structure. The two complete structures were further minimized by heating to 300 K, equilibration for 1 ps, 200 steps of steepest gradient minimization and 10 000 steps of conjugate gradient minimization to optimize the hydrogen bonds, ion pairs and hydrophobic interactions. The minimization was performed by the Discover 3 program with the CVFF forcefield.

To investigate interaction between ligand and receptors, the three-dimensional structures of IL-4R[alpha] and IL-13R[alpha]1 were generated by application of restraint- and structure-based homology modeling techniques (42). The extracellular part of IL-4R consists of two cytokine receptor domains. In contrast, IL-13R[alpha]1 has an additional fibronectin type III domain at its N-terminus. The shorter extracellular sequence of IL-4R[alpha] and comparison with the structures of the human growth hormone (hGH) and the EPOR (erythropoietin receptor) complexes indicate that, in the complex including IL-4R[alpha] 1, IL-13 must bind to domains 2 and 3 of IL-13R[alpha] 1. These two domains also show the cytokine receptor-specific cysteine/tryptophanlproline pattern. The structures of hGHR (43), EPOR (44) and gp130 (45) served as templates for the modeling. The crystal structure of IL-4 bound to IL-4R[alpha] has been published recently (46), but the co-ordinates have not been released yet. However, the information given about loop conformation, residue exposure and inter-protein orientation was taken into account for modeling of IL-4R[alpha]1 and the complex. For IL-13, an earlier prediction was considered (47). The models were refined by initial minimization (5000 steps), short molecular dynamics (40 ps), and final minimization using the Amber forcefield. Functional IL-13R is composed of IL-4R[alpha] and IL-13R[alpha]1 (1-3). Current knowledge about the ‘standard’ structures of Class I cytokine receptor complexes suggests that there are two faces on a cytokine interacting with its two receptor chains. One face is made up of residues predominantly located on helices A and C of the cytokine and interacts with one of the receptor chains. The other face consists of amino acids in the two long loops (loops AB and CD) and helix D of the cytokine. As it was not known whether the AC face of IL-13 interacts in the complex with IL-13R[alpha] 1 or IL-4R[alpha], two models were built. In model I the AC face interacts with IL-4R[alpha], while interacting with IL-13R[alpha]1 in model 2.

Immunohistochemical Assay

Monoclonal antibodies to human IL-13R[alpha]1 were established using the extracellular domain of IL-13R[alpha]1 as antigen. One was designated as UU15 and is an IgG2a isotype. We immunostained human B cells, DND39 cells and human IL-13R[alpha]1-transfected DND39 cells with UU15. It has been confirmed that DND39cells do not express mRNA of human IL-13R[alpha] 1. The transfectants showed strong staining, whereas parental cells did not give rise to any signal. These results confirmed that UU15 recognizes human IL-13R[alpha]1 specifically in immunohistochemistry. Monoclonal antibody to human IL-4R[alpha] was purchased from Genzyme (Cambridge, Mass.). Fresh human lung tissues were obtained and embedded in paraffin from patients undergoing surgery; informed consent was obtained. Asthmatic specimens were obtained from autopsy lungs. The sections were probed with UU15 by the alkaline phosphatase method. Specificity of signal was confirmed by demonstration that: (i) the signal was not elicited by the treatment without the primary antibody; and (ii) adding excess IL-13R[alpha]1 or IL-4R[alpha] to the reaction diminished the signal.

Statistics

Contingency table analysis, ORs, 95% Cis, and significance values were calculated by computerized methods (SPSS program v8). If the number in the column was <10, Fisher's exact method was used. Probability values were corrected for multiple comparison by multiplying the P values by the number of loci compared (Bonferroni correction) ANOVA and multivariate analyses were also performed using this program; two-, three-, four- and five-way step interactions were tested. Linkage disequilibrium was calculated as described (48).

Sequence Database

Sequences used here for modeling were derived from the database at DDBJ/EMBL/GenBank: IL-13 (accession nos U10307, L06801, L13029), IL-4 (M23442), IL-4R[alpha] (X52425), IL-13R[alpha]1 (U62858), IL-13R[alpha]2 (X95302), EPOR (M34986. M60459), GHR (M28466), gp130 (M57230). Note that numberings in this text is on the basis of mature peptides. Protein numbering is based on the cytokine-web (http://www.psynix.co.uk/cytweb/targets/index.html).

Results

Genetic Association Study of an IL-13 Variant in Case-Control Populations

A single-strand conformation polymorphism (SSCP) analysis among >200 atopic subjects identified different electrophoretic patterns in exon 4 of IL-13; subsequent direct sequencing identified an A4464G variant (18), indicating replacement of Arg by Glu at position 110 of the mature protein. Despite intensive screening we could not find any further common variants. We went on to test for a genetic association between Gln110Arg and clinical asthma and IgE levels in two populations (Table 1). The genotype frequencies were concordant with Hardy-Weinberg equilibrium. No significant differences in genotype frequencies were seen between two control populations: P_{Arg}=0.92, P_{Gln}=0.08 in a British population, and P_{Arg}=0.90, P_{Gln}=0.10 in a Japanese population. In a case-control study of British subjects, the Gln110 significantly associated with asthma, especially chronic unremitting asthma. In a second case-control study of Japanese subjects, Gln110 associated with atopic [odds ratio (OR)=1.85, 95% CI: 1.05-3.24, P=0.033] and also non-atopic (‘intrinsic’) asthma (OR=1.77, 95% CI 1.01-3.10, P=0.047); the overall OR for asthma was 1.81 (95% CI 1.11-2.93, P=0.017). There was no association between Gln110 and serum IgE levels in either population.

Genetic Association Study of the IL-13 variant in a general population in relation to serum IL-13 Levels

To test the relationship between the Gln110Arg of IL-13 and serum IL-13 levels, we conducted a population-based survey of genotype frequencies among Japanese schoolchildren aged between 12 and 13 years (19). A significantly higher frequency of the Gln110 allele was seen among asthmatic children than among non-asthmatic subjects. Those homozygous for Gln110 had significantly higher levels of serum IL-13 than those homozygous for Arg110. Genetic association study of an IL-13R[alpha] 1 variant in case-control populations We have searched for single nucleotide polymorphisms (SNPs) by SSCP in the coding region of 1L-13RA1 on the X chromosome (14); all three SNPs discovered were silent and only one of them, A1398G, was relatively common (31). This variant showed marginal association with high IgE levels (OR=2.88, 95% CI 1.11-7.86, P=0.021, Pc=0.06) but not with asthma in the British population; no association was seen with either high IgE levels or asthma in the Japanese population (Table 1). Since male subjects are hemizygous at IL-13RA1, we calculated adjusted OR by sex. In the Japanese population, there was no significant difference in OR (1.66 versus 1.21) between male and female subjects for atopy. In the British population, OR was 3.39 (Fisher's exact test, P=0.015) in males, and 1.10 in female (Fisher's exact test, P=0.680) for atopy.

Genetic Interaction Among Variants of IL-4 and IL-13 signaling

To test whether variants of IL-4 and IL-13 and of their receptor genes, IL-4R or IL-13RA1, might interact in the development of asthma and atopy, we conducted simple factorial analysis of variance (ANOVA): there was no significant genetic interaction between these variants of ligand and receptors in the development of asthma or atopy in either the British or the Japanese populations (Table 2). In the development of asthma, the Gln110 variant of IL-13 is a significant factor in both populations; atopy associates with either an 1IL-13RA1 variant in the British population or an IL-4R variant in the Japanese population (15, 16); in our German population, atopy is also mainly related to IL-4R variants (OR=0.36, P=0.0042 for the combination with Pro478 and Arg551) (17).

Computer Modeling of IL-13 and Its Receptor

To address the biological activity of the Gln 110 variant of IL-13, molecular modeling was conducted on the basis of sequence alignment between IL-4 and IL-13 (20). Firstly, the modeling suggested that the replacement of Arg with Gln at position 110 may allow Arg10 to become closer to Gln110 with electrostatic interaction within IL-13 itself, this may result in subsequent change of electrostatic potential around glutamic acids at positions 11 and 14, and the former is believed to be important for IL-13 binding to the receptor on the basis of IL-4 homology (20).

Further molecular modeling focused on the interaction of IL-13 with IL-4R[alpha] and IL-13R[alpha]1, and was conducted on the basis of multiple alignment of cytokine receptors. Two alternative models showed that Arg 110 is likely to have direct interaction with one or other of the component receptor chains. Closer inspection of Arg 110 in model I showed that this residue was located in proximity to His131 of IL-4R[alpha]. As both residues are positively charged, this predicted a repulsive interaction. A Gln110 mutant would abolish this expulsion and, thus, enhance receptor binding of IL-13 with consequent upregulation of IL-13 signaling. In model 2, Arg 110 lies close to Glu267 and Val270 of IL-13R[alpha] 1, implying an attractive interaction. A Gln110 mutant would lead to reduced binding. In the light of the association of Gln110 with asthma, model 1 may be more plausible. However, model 2 remains interesting since such a mutation might lead to an alternative binding mode or induce reversal of model 2 binding mode to a model 1 binding mode. In either case, computer modeling strongly suggests that Arg 110 is directly involved in interactions with its receptor, and that charge changing variants (Arg, Gln) are likely to display different biological properties.

Immunohistochemical Assay With Bronchial Specimens

Only in murine studies has the existence of IL-13R been demonstrated in airways (1.2). Therefore, we conducted immunohistochemistry on pulmonary specimens from normal controls and asthmatic subjects, using monoclonal antibodies to both IL-13R[alpha]1 and IL-4R[alpha]. Both components were present prominently in smooth muscle cells and epithelial cells of the bronchus, but not in fibroblasts. Within alveolar tissue, neither epithelial cells nor pulmonary macrophages showed either component. Specificity of signal was confirmed by demonstration that the signal was not elicited by the treatment without the primary antibody. Thus, the data suggest that functional IL-13R is specifically expressed in epithelial and muscle cells of the human bronchus.

Discussion

Our data suggest an important role for genetic variants of IL-13 in the development of asthma, independently of IL-4, in humans. Many groups have identified linkage of both asthma and IgE levels to chromosome 5q31(21, 22), where IL-4, IL-13 and IL-5 cluster is localized. Although IL-4 is crucial for the development of Th² cells (1-3), and hence high IgE levels (23-26), IL-4 may not be a sufficient inducer for asthma itself. No functional polymorphism in IL-5 has been identified in relation to either asthma (27) or eosinophilia (28).

We have identified a novel coding variant of the human IL-13, Gln110Arg, that associates with asthma in both British and Japanese populations. Our computer modeling suggests that Gln at position 110 impacts on ligand-receptor interaction, through enhanced charge attraction to IL-13R. This in turn may enhance signaling, but detailed functional studies are now needed to test this. The genetic association of IL-13 with asthma across different ethnic populations, which is independent of IL-4, supports the candidacy of IL-13 as a major locus for asthma on chromosome 5q3l. Moreover, we found a genetic association of the Gln110Arg variant of IL-13 with both atopic and non-atopic (intrinsic) asthma-a finding concordant with clinical reports on the significant elevation of IL-13 levels in both types of asthmatic subject (11, 12). These findings extend the observation of association between a promoter polymorphism of IL-13 and allergic asthma in a Dutch population (13), and support the contention that IL-13 may be a key promoter of bronchial asthma in humans.

There is strong evidence that IL-13 is crucial for the induction of an asthma-like phenotype in animal models, independent of IL-4 (9, 10), but which is dependent on IL-4R[alpha], a common component of IL-4R and IL-13R. T cell-deficient mice are capable of inducing an asthma-like phenotype on administration of IL-13 (9), suggesting that IL-13 may operate through mechanisms other than those that are classically implicated in Th² cell-induced immune reactions (10). One possible explanation is that IL-13R is predominantly expressed in bronchial tissues, and that higher production of IL-13 in high risk genotypes induces hypertrophic change of the bronchial smooth muscle, subepithelial fibrosis and goblet cell hyperplasia through IL-13R. To investigate this possibility, we stained airway specimens from normal controls and asthmatic subjects with anti-IL-13R[alpha] 1 and anti-IL-4R[alpha] monoclonal antibodies. In asthmatic airways, in contrast to alveolar tissue, both components of IL-13R are significantly expressed. To date, two types of specific IL-13 receptor unit have been identified: IL-13R[alpha]1 (29) and IL-13R[alpha]2 (30). IL-13R[alpha]2 is considered to be a ‘decoy’ receptor (31, 32), whereas the heterodimer consisting of IL-I 3R[alpha] 1 and IL-4R[alpha] acts as the functional receptor for human IL-13 (12). Human IL-4R[alpha] is constitutively expressed in airway epithelial cells (33, 34), and is essential for mucus production (7-9). Although human IL-4 induces goblet cell hyperplasia in vivo (35), Th² cells derived from IL-4-null mice cannot activate goblet cells even after transfection into IL-4R[alpha]-null mice, suggesting that, in the absence of IL-4, IL-13 may be critical for goblet cell activity through IL-13R in airways (7-9). Also, IL-13 transgenic mice show BHR to methacholine, and subepithelial fibrosis, whereas IL-4 transgenic mice do not (8). The immunohistochemical findings in human lung specimens suggest that functional IL-13R is expressed in bronchial tissues in asthmatic subjects, and support the idea that IL-13 may play a key role in the development of asthma.

IL-13 and IL-4 have overlapping effector profiles (1-3). This overlap is probably due to shared use of IL-4R[alpha], and the IL-13R[alpha] 1 chains in the multimeric receptor complex (1-3). Chromosome 16p11.2/12, where the IL-4R[alpha] gene is encoded, has also been shown to be linked to atopy (36). We have previously identified a common extracellular variant, Ile50Val of the IL-4R[alpha], which associates strongly with atopic asthma in the Japanese but not in the British population (Table 1) (15, 16). Furthermore, we showed that Ile50 strongly and specifically associated with atopic, rather than intrinsic or non-atopic, asthma: it up-regulated cellular IgE synthesis when tested by transfection into B cell lines. Another cytoplasmic variant of IL-4R[alpha], Arg551Gln, associates with atopy (17); in our German population this variant was in tight linkage disequilibrium with Pro478Ser and the latter may change the structure of the receptor, leading to altered phosphorylation patterns of signal molecules, and hence lower IgE levels (17).

We now show that a variant of IL-13RA1 on the X chromosome (14) showed association with IgE levels, but not with asthma, in British male subjects; significantly, the OR for high IgE levels was 3.38 in males, but 1.10 in females. This is the first indication of X-linked inheritance of an atopic immune disorder with high IgE levels, and further illustrates the heterogeneity of its genetic basis. It may also, in part, explain the previously noted transmission of atopy through non-affected mothers (37, 38). The functional role of this variant remains unknown, it may be in linkage disequilibrium with so far unidentified polymorphisms in the regulating or coding parts of IL-13RA1 or variants of IL-13RA2 (30) close by on the X chromosome (14). Further studies are needed to clarify these points.

In conclusion, we have identified a novel coding variant of the human IL-13, Gln110Arg, on chromosome 5q31 where genome-wide searches have identified linkage to asthma (23, 24). Gln110Arg shows association with clinical asthma across different ethnic populations, including atopic (high IgE levels) and non-atopic asthma (Table 1): it associates with higher serum IL-13 levels. Immunohistochemical techniques revealed that both components of IL-13R are present in epithelial and smooth muscle cells of the human bronchus in asthma. Molecular modeling points to modification of ligand-receptor interaction by Gln110Arg substitution with increased ligand-receptor attraction. We therefore propose that Gln110Arg IL-13, the product of effector cells in asthmatics, promotes smooth muscle hyper-reactivity and mucus hyper-secretion through IL-13R in human bronchi and, thus, promotes clinical asthma. Our observations and conclusions are consistent with the observed central role of IL-13 in experimental asthma in animal models (9, 10); detailed functional analyses on the variant are now required. In contrast, we have found that high IgE levels associate with variants of IL-4R[alpha] or IL-113R[alpha] 1, with restriction amongst different ethnic groups (Table 1). A multivariate analysis shows that the associations are independent among the variants of IL-4, IL-13, IL-4R[alpha] and IL-13R[alpha] 1 (Table 2). The implication of variants of IL-4 and IL-13 signaling in the development of asthma and atopy in humans provides a focus for considering novel therapeutic and preventive strategies.

TABLE 1
Odds ratio (95% CI) for IL-13 and receptor genes, IL-4R and IL-13RA1, by asthma, total serum IgE, ASE and atopy in the Japanese and the British populations

| | IL-13 | | | IL-4Rα | | | | | | IL-13α1 | | |
|---|---|---|---|---|---|---|---|---|---|---|---|---|
| | Gln110Arg | | | Ile50Val | | | Arg55Gln | | | A1398G | | |
| | % Gln | OR (95% CI) | P value | % Ile/Ile | OR (95% CI) | P value | % Arg(+) | OR (95% CI) | P value | % A/A | OR (95% CI) | P value |
| Japanese | | | | | | | | | | | | |
| Controls | 43 | | | 18.0 | | | 22.0 | | | 40.0 | | |
| Atopic asthma | 63 | 1.85 (1.05-3.24) | 0.033 | 59.0 | 4.42 (1.57-6.69) | <0.0001 | 32.0 | 1.66 (0.88-3.13) | 0.111 | 33.0 | 1.35 (0.76-2.41) | 0.304 |
| Non-atopic asthma | 62 | 1.77 (1.01-3.10) | 0.047 | 18.0 | 1.00 | 1.00 | 24.0 | 1.12 (0.58-2.17) | 0.737 | 36.0 | 1.19 (0.67-2.10) | 0.56 |
| Asthmatics | 62.5 | 1.81 (1.11-2.93) | 0.013 | 38.5 | 1.52 (0.68-2.98) | <0.151 | 28.0 | 1.38 (0.77-2.78) | 0.264 | 34.5 | 1.27 (0.77-2.08) | 0.350 |
| Low serum IgE | 56.3 | | | 17.6 | | | 23.5 | | | 38.6 | | |
| High serum IgE | 60.2 | 1.18 (0.72-1.90) | 0.508 | 53.5 | 3.09 (1.98-4.91) | <0.0001 | 28.5 | 1.28 (0.81-2.02) | 0.288 | 32.4 | 1.18 (0.72-1.93) | 0.516 |
| ASE (−) | 52.7 | | | 18,2 | | | 23.6 | | | 38.9 | | |
| ASE (+) | 63.9 | 1.59 (1.00-2.53) | 0.051 | 52.3 | 3.17 (1.98-5.07) | <0.0001 | 28.0 | 1.26 (0.63-2.52) | 0.319 | 33.1 | 1.29 (0.80-2.08) | 0.296 |
| Non-atopic | 52.3 | | | 17.0 | | | 23.3 | | | 40.1 | | |
| Atopic | 64.6 | 1.66 (1.01-2.57) | 0.047 | 62.4 | 7.04 (2.08-9.88) | <0.0001 | 28.8 | 1.59 (0.91-3.03) | 0.093 | 31.9 | 1.28 (0.80-2.08) | 0.305 |
| British | | | | | | | | | | | | |
| Controls | 26.7 | | | 34.6 | | | 41.3 | | | 4.0 | | |
| Atopic asthma | 40.0 | 1.83 (1.13-2.99) | 0.014 | 32.0 | 1.12 (0.81-2.34) | 0.624 | 35.5 | 0.78 (0.49-1.24) | 0.342 | 12.0 | 3.22 (1.26-8.33) | 0.018 |
| Non-asthmatics | 23.5 | | | 31.3 | | | 35.2 | | | 4.2 | | |
| Asthmatics | 39.8 | 2.14 (1.28-3.60) | 0.003 | 34.3 | 1.11 (0.68-1.82) | 0.770 | 32.4 | 0.73 (0.45-1.17) | 0.236 | 10.5 | 2.70 (0.98-7.14) | 0.053 |
| Severe asthmatics | 41.5 | 2.31 (1.33-4.00) | 0.003 | 36.3 | 1.39 (0.74-2.13) | 0.488 | 33.8 | 0.78 (0.47-1.30) | 0.409 | 10.0 | 2.50 (0.87-7.15) | 0.090 |
| Low serum IgE | 29.3 | | | 31.3 | | | 37.7 | | | 3.6 | | |
| High serum IgE | 38.3 | 1.49 (0.92-2.42) | 0.100 | 36.3 | 1.25 (0.30-1.47) | 0.366 | 39.1 | 1.06 (0.66-1.69) | 0.902 | 13.5 | 2.88 (1.11-7.86) | 0.021* |
| ASE (−) | 30.0 | | | 31.2 | | | 39.2 | | | 4.5 | | |
| ASE (+) | 38.3 | 1.27 (0.77-2.10) | 0.351 | 34.9 | 1.05 (0.29-1.28) | 0.856 | 37.5 | 0.62 (0.39-1.01) | 0.071 | 10.0 | 2.32 (0.85-6.45) | 0.122 |
| Non-atopic | 27.2 | | | 30.2 | | | 35.2 | | | 4.0 | | |
| Atopic | 36.3 | 1.52 (0.90‥2.58) | 0.118 | 37.3 | 1.23 (0.73-2.04) | 0.515 | 36.5 | 0.82 (0.50-1.34) | 0.520 | 10.0 | 2.63 (0.87-7.69) | 0.111 |

*OR in females = 1.10 (95% CI:0.81-2.81, Fisher's exact test, P = 0.765), in males = 3.39 (95% CI 1.04-9.35, Fisher's exact test, P = 0.015 (see text).

TABLE 2
Simple factorial ANOVA analysis for genetic variants in the development of
asthma and atopy in the British and the Japanese populations

| | | | F value (df = 2) | | | |
|---|---|---|---|---|---|---|
| Phenotype | Genetic factor | Variant | British | P value | Japanese | P value |
| Asthma | IL-4 | −590C/T | 0.511 | 0.600 | 0.490 | 0.623 |
| | IL-13 | Gln110Arg | 4.133 | 0.017 | 4.283 | 0.026 |
| | IL-4Rα | Ile50Val | 2.511 | 0.083 | 2.614 | 0.076 |
| | | Arg551Gln | 1.121 | 0.328 | 0.998 | 0.344 |
| | IL-13Rα1 | A1398G | 2.704 | 0.069 | 1.134 | 0.313 |
| Atopy (IgE levels) | IL-4 | −590C/T | 0.229 | 0.795 | 0.748 | 0.475 |
| | IL-13 | Gln110Arg | 0.935 | 0.394 | 1.494 | 0.206 |
| | IL-4Rα | Ile50Val | 0.362 | 0.697 | 5.272 | 0.006 |
| | | Arg551Gln | 1.242 | 0.290 | 0.206 | 0.814 |
| | IL-13Rα1 | A1398G | 4.288 | 0.015 | 0.829 | 0.479 |

We found no linkage disequilibrium between the IL-4 (−590C/T) and the IL-13 (Gln110Arg) variants in either the British (t = Δ/SE = −0.43) or the Japanese (t = Δ/SE = −1.29) population.

The following references are cited throughout the specification and are incorporated herein by reference.

1. Izuhara et al (1999) Signal transduction via the interleukin-4 receptor and its correlation with atopy. Int. J. Mol. Med. 3:3-10.

2. Shirakawa et al (2000) Asthma, atopy and genetic variants of IL-4 and IL-13 signaling. Immunol. Today 21:60-64.

3. Nelms et al (1999) The interleukin-4 receptor: signal mechanisms and biologic functions. Ann. Rev. Immunol. 16:701-736.

4. Yamaguchi et al (1988) Purified interleukin 5 supports the terminal differentiation and proliferation of murine eosinophilic precursors. J. Exp. Med. 167:1737-1742.

5. Foster et al (1996) Interleukin 5 deficiency abolishes eosinophilia, airways hyperreactivity, and lung damage in a mouse asthma mode. J. Exp. Med. 183:195-201.

6. Hogan et al (1998) A novel T cell-regulated mechanism modulating allergen-induced airways hyperreactivity in BALBIc mice independently of IL-4 and IL-5. J. Immunol. 161:1501-1509.

7. Bancroft et al (1998) A critical role for IL-13 in resistance to intestinal nematode infection. J. Immunol. 160:3453-3461.

8. Zhu et al (1999) Pulmonary expression of interleukin-13 causes inflammation, mucus hypersecretion, subepithelial fibrosis, physiologic abnormalities, and eotaxin production. J. Clin. Invest. 103:779-788.

9. Grunig et al (1998) Requirement for IL-13 independently of IL4 in experimental asthma. Science 282:2261-2263.

10. Wills-Karp et al (1998) Interleukin-13: central mediator of allergic asthma. Science 282:2258-2261.

11. Humbert et al (1997) Elevated expression of messenger ribonucleic acid encoding IL-13 in the bronchial mucosa of atopic and nonatopic subjects with asthma. J. Allergy Clin. Immunol. 99:657-665.

12. Huang et al (1995) IL-13 expression at the sites of allergen challenge in patients with asthma. J. Immunol. 155:2688-2694.

13. van der Pouw Kraan et al (1999) An IL-13 gene promoter promoter polymorphism associated with increased risk of allergic asthma. Genes Immunity 1:61-65.

14. Guo et al (1997) Chromosomal mapping and expression of the human interleukin-13 receptor. Genomics 42:141-145.

15. Mitsuyasu et al (1998) Ile50Val variant of the IL4R upregulates IgE synthesis and associates with atopic asthma Nature Genet. 19:119-120.

16. Mitsuyasu et al (1999) Dominant effect of Ile50Val variant of the human IL-4 receptor [alpha]-chain in IgE synthesis. J. Immunol. 162:1227-1231.

17. Kruse et al (1999) The polymorphisms S503P and Q576R in the interleukin-4 receptor [alpha] gene are associated with atopy and influence signal transduction. Immunol. 96: 365-371.

18. Smimov et al (1995) Tandem arrangement of human genes for interleukin-4 and interleukin-13: resemblance in their organisation. Gene 155:277-281.

19. Shirakawa et al (1997) The inverse association between tuberculin response and atopic disorder. Science 275:77-79.

20. Zurawski et al (1993) Receptors for interleukin-13 and interleukin-4 are complex and share a novel component that functions in signal transduction. EMBO J. 12:2663-2670.

21. Marsh et al (1994) Linkage analysis of IL-4 and other chromosome 5q31.1 markers and total serum immunoglobulin E concentrations. Science 264:1152-1156.

22. Postma et al (1995) Genetic susceptibility to asthma-bronchial hyperresponsiveness coinherited with a major gene for atopy. N. Engl. J. Med. 333:894-900.

23. The Collaborative Study on the Genetics of Asthma (1997) A genome-wide search for asthma susceptibility loci in ethnically diverse populations. Nature Genet. 15:389-392.

24. Ober et al (1998) Genome-wide search for asthma susceptibility loci in a founder population. The Collaborative Study on the Genetics of Asthma. Hum. Mol. Genet. 7:1393-1398.

25. Rosenwasser et al (1997) Genetics of atopy and asthma: the rationale behind promoter-based candidate gene studies (IL-4 and IL-10). Am. J Respir. Crit. Care Med. 156: S152-S155.

26. Noguchi et al (1998) Association of asthma and the interleukin-4 promoter gene in Japanese. Clin. Exp. Allergy 28:449-453.

27. Pereira et al (1998) Mutation analysis of interleukin-5 in an asthmatic cohort. Hum. Mutat. 11:51-54.

28. Rioux et al (1998) Familial eosinophilia maps to the cytokine gene cluster on human chromosomal region 5q31-q33. Am. J. Hum. Genet. 63:1086-1094.

29. Aman et al (1996) cDNA cloning and characterization of the human interleulkin 13 receptor [alpha] chain. J. Biol. Chem. 271:29265-29270.

30. Caput et al (1996) Cloning and characterization of a specific interleukin (IL)-13 binding protein structurally related to the IL-5 receptor [alpha] chain. J. Biol. Chem. 271:16921-16926.

31. Donaldson et al (1998) The murine IL-13 receptor [alpha]2: molecular cloning, characterization, and comparison with murine IL-13 receptor [alpha] 1. J. Immunol. 161:2317-2324.

32. Lutz et al (1999) Binding of interleukin-13 and interleukin-4 to the interleukin (IL)4/IL-13 receptor of human synovial fibroblasts. J. Recept. Signal Transduct. Res. 19:181-190.

33. van der Velden et al (1998) Interleukin 4 receptors on human bronchial epithelial cells, an in vivo and in vitro analysis of expression and function. Cytokine 10:803-813.

34. Kotsimbos et al (1998) Expression of the IL-4 receptor [alpha]-subunit is increased in bronchial biopsy specimens from atopic and nonatopic asthmatic subjects. J. Allergy C/in. Immunol. 102:859-866.

35. Dabbagh et al (1999) IL-4 induces mucin gene expression and goblet cell metaplasia in vitro and in vivo. J. Immunol. 162:6233-6237.

36. Deichmann et al (1998) Linkage and allelic association of atopy and markers flanking the IL4-receptor gene. Clin. Exp. Allergy 28:151-155.

37. Shirakawa et al (1994) Association between atopy and variants of the [beta] subunit of the high-affinity immunoglobulin E receptor. Nature Genet. 7:125-129.

38. Sandford et al (1993) Localisation of atopy and [beta] subunit of high-affinity IgE receptor (Fc[epsiv]RI[beta]) on chromosome 11q. Lancet 341:332-334.

39. Gao et al (1998) Negative association between asthma and variants of CC 16 (CC10). Hum Genet. 103:57-59.

40. Mao et al (1996) Association between genetic variants of mast-cell chymase and eczema. Lancet 348:581-583.

41. Walley et al (1996) Investigation of an interleukin-4 promoter polymorphism for associations with asthma and atopy. J. Med. Genet. 33:689-692.

42. Blundell et al (1987) Knowledge-based prediction of protein structures and the design of novel molecules. Nature 326:347-352.

43. deVos et al (1992) Human growth hormone and extracellular domain of its receptor: crystal structure of the complex. Science 255:306-312.

44. Livnah et al (1996) Functional mimicry of a protein hormone by a peptide agonist: the EPO receptor complex at 2.8 Å. Science 273:464471.

45. Bravo et al (1998) Crystal structure of a cytokine-binding region of gp 130. EMBO J. 17:1665-1674.

46. Hage et al (1999) Crystal structure of the interleukin 4/receptor [alpha] chain complex reveals a mosaic binding interface. Cell 97:271-281.

47. Bamborough et al (1994) Predictive modeling of the 3-D structure of interleukin-13. Protein Eng 7: 1077-1082.

48. Mittal (1976) The HLA polymorphism and susceptibility to disease. Vox. Sang. 31:161-173.

49. Gergen et al (1992) Am. Rev. Respir. Dis. 146:823-824.

50. Marsh et al (1982) N. Engl. J. Med. 305:1551-1559.

51. Clifford et al (1987) Arch. Dis. in Childhood 62:66-73.

52. Burrows et al (1989) N. Engl. J Ved. 320:271-277.

53. Goodman and Gilman's The Pharmacologic Basis of Therapeutics, Seventh Ed., MacMillan Pub. Co., NY (1985).

54. Alexander et al (1992) Lancet 339:324-328).

55. Chu et al (1992) Cell Immunol. 145:223-239.

56. Zav′yalov et al (1992) Immunol. Lett. 31:285-288.

57. Gessner et al (1993) Immunobiology 189:419-435.

58. Burrows et al (1992) J. Allergy Clin. Immunol. 90:376-385.

59. Hopp et al (1988) Ann. Allergy 61:184-186.

60. Ackerman et al (1994) Chest 105:687-696.

61. Sears et al (1991) N. Engl. J. Med. 325:1067-1071.

62. O'Connor et al (1989) Am. Rev. Respir. Dis. 140:225-252.

63. Cookson et al (1989) Lancet 1292-1295.

64. Moffatt et al (1992) Clin. Exp. Allergy 22:1046-1051.

65. Amelung et al (1992) Clin. Exp. Allergy 22:1077-1084.

66. Marsh et al (1974) Proc. Natl. Acad. Sci. USA 71:3588-3592.

67. Blumenthal et al (1981) Am. J. Med. Genet. 10:219-228.

68. The Genome Data Base. The Welch Library, The John Hopkins Med. Inst., Baltimore, Md.

69. Marsh et al (1994) Science 264:1152-1156.

70. Postma et al (1995) N. Engl. J. Med. 333:894-900.

71. Meyers et al (1994) Genomics 23:464-470.

What is claimed is:

1. A method for detecting a predisposition to asthma or atopy in an individual, the method comprising: analyzing a biological specimen from said individual for the presence of a Il-13 Gln110Arg variant; wherein the presence of said variant is indicative of an increased susceptibility to asthma or atopy.

2. The method of claim 1, wherein the biological specimen is genomic DNA or mRNA.

3. A method according to claim 2, wherein said analyzing step comprises detection of specific binding between the genomic DNA or mRNA of said individual with a probe comprising nucleic acid encoding Gln110Arg IL-13 variant or a fragment thereof.

4. An isolated nucleic acid molecule encoding a Gln110Arg IL-3 variant.

5. The isolated nucleic acid molecule according to claim 4 wherein said nucleic acid comprises a promoter or regulatory region.

6. The isolated nucleic acid molecule according to claim 4 comprising a probe for detection of an Gln110Arg IL-13 variant.

7. An array of oligonucleotides comprising: a probe according to claim 6.

8. A cell comprising a nucleic acid composition according to claim 4.

9. A non-human transgenic animal model for Gln110Arg IL-13 gene function comprising an exogenous and stably transmitted mammalian Gln110Arg IL-13 gene sequence.

10. A method of screening for biologically active agents that modulate Gln110Arg IL-13 function, the method comprising:
combining a candidate biologically active agent with any one of:
(a) Gln110Arg IL-13 polypeptide;
(b) a cell comprising a nucleic acid encoding Gln110Arg IL-13 variant polypeptide; or
(c) a non-human transgenic animal model for Gln110Arg gene function comprising an exogenous and stably transmitted mammalian Gln110Arg IL-13 gene sequence and determining the effect of said agent on Gln110Arg IL-13 variant function.
